(19) United States
(12) Patent Application Publication
(10) Pub. No.: US 2009/0156883 A1
(43) Pub. Date: Jun. 18, 2009

(54) ELECTROMAGNETIC FORCE FOR ENHANCING TISSUE REPAIR

(76) Inventors: Thomas J. Goodwin, Kemah, TX (US); Clayton R. Parker, Safety Harbor, FL (US)

Correspondence Address: LADAS & PARRY LLP, 224 SOUTH MICHIGAN AVENUE, SUITE 1600, CHICAGO, IL, 60604, US

(21) Appl. No.: 11/993,895

(22) PCT Filed: Jun. 23, 2006
(86) PCT No.: PCT/US06/24814

§ 371 Date: Nov. 13, 2008

Related U.S. Application Data
(63) Continuation-in-part of application No. 11/169,614, filed on Jun. 29, 2005, now Pat. No. 7,179,217.

(51) Int. Cl. A61N 2/04

(52) U.S. Cl. 600/14

(57) ABSTRACT

A time varying electromagnetic force sleeve (10) comprising a time varying electromagnetic force source (3) operatively connected to a coil (1) having a conductive portion (7), a coil support (5), and an interior portion wherein the interior portion defines a space that removably receives a mammalian body part. Also provided is a method for enhancing repair of defective tissue of a mammal.

### FIELD OF THE INVENTION

The present invention relates generally to sleeves, and more particularly to a time varying electromagnetic force sleeve that has an interior portion that removably receives a mammalian body part. The present invention also relates to a time varying electromagnetic force sleeve that, in use, can enhance tissue repair.

### BACKGROUND OF THE INVENTION

The power of the magnet is one of the most basic powers in nature. Magnetism itself was an ingredient in the primordial soup from which the universe and the planet came forth. Magnetism is the force that keeps order in the galaxy, allowing stars and planets to spin at significant velocities.

Magnetic therapy has long been the subject of controversy. Many veterinarians have been aware of bio-magnetic benefits for years and use magnets to heal fractures quickly thereby saving the lives of racehorses and other animals. Furthermore, doctors treating professional athletes commonly recommend magnets to speed up recovery from painful injuries. Other doctors in a variety of specialties, including dermatologists, internists, pediatricians, and surgeons, have used magnets with varying claims of success.

The restorative properties of magnetic therapy have long been known and relied on by early scientifically advanced civilizations that have documented the same. Ancient Greece discovered the very first natural magnet in the form of the lodestone. Hippocrates, the father of medicine, noted the lodestone's healing powers. The Egyptians described the divine powers of the magnet in their writings, and Cleopatra frequently adorned herself with magnetic jewelry to preserve youthfulness. Chinese manuscripts dating back thousands of years describe the Eastern belief that the life force, termed “qi,” is generated by the earth's magnetic field. Today, many believe that certain places on earth, such as Lourdes, France, and Sedona, Ariz., owe their healing powers to naturally high levels of this qi, or bio-magnetic energy.

Magnetic therapy is used in many countries such as Japan, China, India, Austria, and Germany. Although state-of-the-art American medicine uses techniques to monitor magnetic fields, such as electrocardiograms, electroencephalograms, and magnetic resonance imaging, it has not taken other forms of magnetic therapy seriously. However, American studies are more and more considering whether or not magnetic therapy has medicinal value. As a result, increasing numbers of people are sleeping on magnetic beds at night and wearing small magnets during the day for greater energy, preventive purposes, and healing with varying degrees of success.

Research into magnet therapy is divided into two distinct areas: pulsed bioelectric magnetic therapy and fixed magnetic therapy. Probably 85 to 90 percent of the scientific literature is on pulsed bioelectric bio-magnetic therapy; the remainder is on therapy with fixed solid magnets. There are different schools of thought on the essential mechanisms of magnetic therapy centered on questions of polarity among other issues. However, fixed magnetic therapy has yet to be widely accepted by the scientific and medical community.

The effectiveness of using pulsed magnetic fields to heal bone fractures and, to lesser degree, soft tissue injuries such as sprains and strains, has been debated for some time. Multiple theories have been advanced to explain electromagnetic healing of many ailments, including osteoarthritis, rheumatoid arthritis, fibromyalgia, tension headaches, migraines, and Parkinson's disease. Numerous scientific journals have reported these findings since the 1970s. Moreover, the FDA approves the use of pulsed electromagnetic fields for the treatment of nonunion bone fractures, which are fractures that will not heal on their own. It is believed that pulsed electromagnetic fields penetrate the cast and get to the layer of skin that's moist and conductive, where the electric field stops, but the magnetic field continues to do the healing work.

Most of the prior attempts to use electromagnetic therapy have used high levels of electromagnetism, usually 50 gauss or more. While most of this therapy has used flat magnetic generators, a few have wrapped a magnetic blanket around a body member to attempt to regenerate or heal the body part. Some of the attempts have used pulsed waves, but such pulsed waves have been either on-off pulses or sinusoidal waves.

Most recently, Simon et al. (U.S. Pat. Publ. No. US2006/0030896 A1) specifically disclose a device and method for using the device to treating degenerative disc disease. The method in Simon et al. incorporate a coil, but only at the site of a degenerated disc. In U.S. Pat. Publ. No. US2006/0030895 A1, Simon et al. disclose a method for treating degenerative disc disease by identifying a disc of interest and electrically stimulating the disc with electrical signals in different waveforms. Furthermore, in U.S. Pat. Publ. No. US2006/0030895 A1, Simon et al. further disclose a method for treating degenerative disc disease by the use of two electrodes placed on the body and delivering voltage in different wave forms. Moreover in U.S. Pat. Publ. No. US2006/0057693 A1, Simon et al. disclose a method of treating a tissue defect using mesenchymal stem cells by administering an electrical stimulation to the mesenchymal stem cells in vitro, and another method of doing the same by implanting the mesenchymal cells at the site of interest, and then applying an electrical stimulation to the cells in vivo.

Consequently, it would be highly desirable to have a time varying electromagnetic force (“TVEMF”) sleeve comprising a coil and a TVEMF source operatively connected to the coil, wherein the coil has a conductive portion, a coil support, and an interior portion which defines a space in which a mammalian body part having defective tissue is removably received. It would also be highly desirable to have a TVEMF sleeve that can be introduced to a mammalian body part having defective tissue so that, in use, a TVEMF can be supplied to the defective tissue to enhance repair of the same. The present invention overcomes the problems associated with past and current methods for regenerating tissue, and presents advantages not before seen.

### SUMMARY OF THE INVENTION

The present invention relates to TVEMF sleeve comprising a coil and a TVEMF source operatively connected to the coil wherein the coil comprises a coil support, a conductive portion, and an interior portion wherein the interior portion defines a space wherein a mammalian body part is removably received.

The present invention also relates to a method for enhancing tissue repair comprising the steps of providing a TVEMF sleeve having a coil and a TVEMF source operatively connected to the coil wherein the coil comprises a conductive portion, a coil support, and an interior portion wherein the interior portion defines a space that removably receives a mammalian body part, introducing the TVEMF sleeve to the mammalian body part having defective tissue, and delivering a TVEMF to the mammalian body part having defective tissue to enhance the repair of the defective tissue.

Other aspects, features, and advantages of the present invention will be apparent from the following description of the preferred embodiments of the invention given for the purpose of disclosure. This invention may be more fully described by the preferred embodiment(s) as hereinafter described, but is not intended to be limited thereto.

### BRIEF DESCRIPTION OF THE DRAWINGS

In the drawings,

FIG. 1 is an elevated side view of a TVEMF sleeve.

FIG. 2 is a cross-sectional elevated front view of a TVEMF sleeve.

FIG. 3 is an elevated side view of a TVEMF sleeve.

FIG. 4 is a cross-sectional elevated front view of a TVEMF sleeve.

FIG. 5 is a side perspective of a TVEMF sleeve.

FIG. 6 is a cross-sectional elevated front view of a TVEMF sleeve.

FIG. 7 is a cross-sectional elevated side view of a TVEMF sleeve.

### DETAILED DESCRIPTION OF THE DRAWINGS

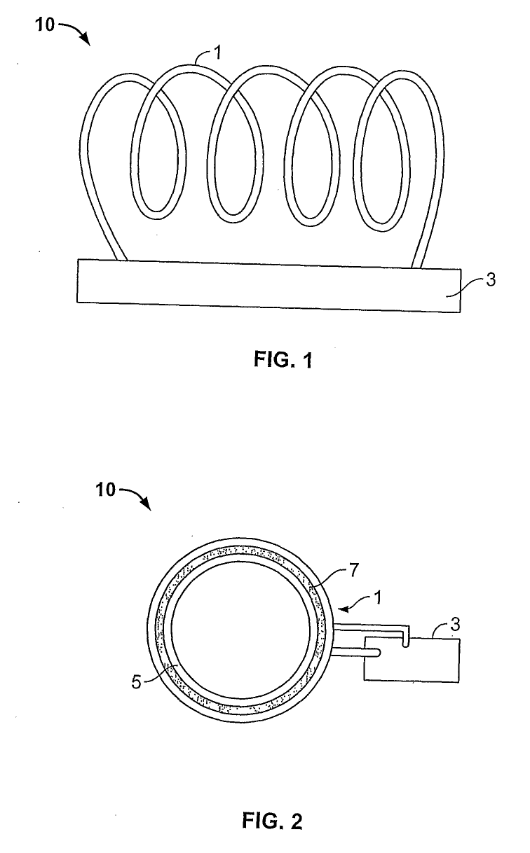

In the drawings, referring now to FIG. 1, illustrated is an elevated side view of a preferred embodiment of a TVEMF sleeve 10 comprising a coil 1 and a TVEMF source 3 operatively connected to the coil 1. The phrase “operatively connected,” and similar words and phrases, is intended to mean that the TVEMF source can be connected to the coil in a manner such that when in operation, the TVEMF source can impart a TVEMF to the coil through a conductive connection, preferably at least one wire. The TVEMF source may preferably be integral with the coil and preferably be affixed to the coil, more preferably removably affixed to the coil. The TVEMF source of the present invention may be commonly found in a hardware store and may preferably be operated with a battery, and/or preferably operated by removably connecting it to an electricity source. The coil of the present invention has an interior portion that defines a space that removably receives a mammalian body part. The space of the interior portion of the coil has a shape, preferably with an elliptical cross-section, more preferably an oval cross-section, and most preferably a circular cross-section. The coil may also preferably be a solenoid, a tightly wound coil.

The phrase, “removably receive,” and any similar terms and phrases, is intended to refer to a characteristic of a space wherein a mammalian body part can preferably be introduced thereto and removed there from as desired. For example, a mammalian body part is removably received by the space in the interior portion of the coil. The mammalian body part can be removed from the space of the interior portion of the coil of the TVEMF sleeve as desired. Furthermore, the term, “introduce,” and similar terms, is intended to mean that the TVEMF sleeve may be introduced to the mammalian body part by being wrapped around, encompassing, fitted to, and/or fitted on the mammalian body part. It is also contemplated that a mammalian body part may be introduced to the TVEMF sleeve by inserting the mammalian body part into the space of the interior portion of the coil of the TVEMF sleeve.

FIG. 2 illustrates a cross-sectional elevated front view of the same preferred embodiment of the TVEMF sleeve 10 depicted in FIG. 1. FIG. 2 shows a TVEMF sleeve 10 with a coil 1 and a TVEMF source 3. The coil 1 comprises a conductive portion 7 and a coil support 5. The conductive portion 7 is preferably an electrically conductive wire that is preferably flexible, and more preferably substantially rigid. The conductive portion may also preferably comprise salt water. In the preferred embodiment illustrated in FIG. 2, the coil support 5 preferably contains the conductive portion 7 and provides insulative characteristics thereto, is preferably flexible, or preferably substantially rigid. The coil support in FIG. 2 may preferably comprise any material that is non-conductive, preferably plastic. Preferably, when the conductive portion 7 is substantially flexible, the coil support is substantially rigid. By “substantially rigid,” it is meant that the coil and/or the coil support can maintain a shape without the need for additional support, that the coil and/or coil support is resistant to a change in the shape. By “substantially flexible,” it is meant that the coil and/or coil support are pliable and the shape of the coil and/or coil support is capable of being changed. The conductive portion of the coil of the present invention may preferably be any conductive material, preferably ferromagnetic, more preferably silver.

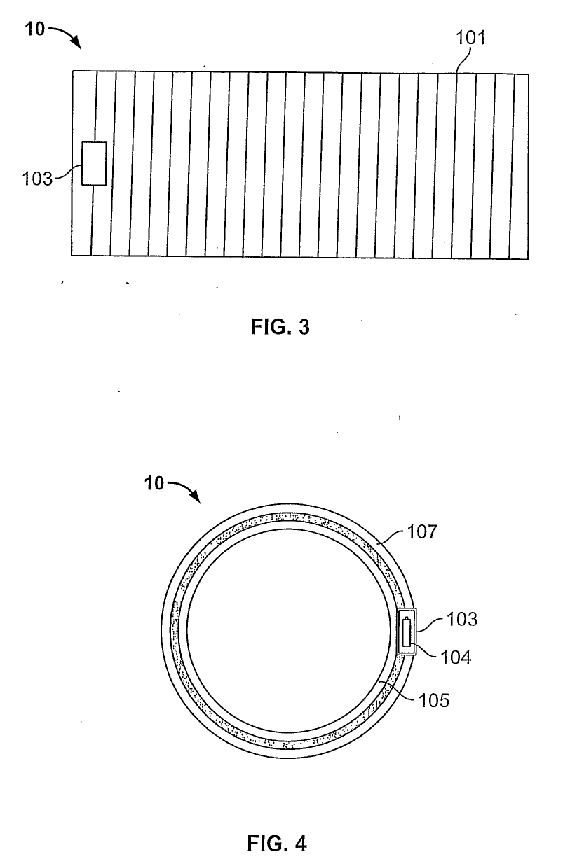

In FIG. 3 is illustrated an elevated side view of another preferred embodiment of the TVEMF sleeve 10 comprising a coil 101 and a TVEMF source 103 operatively connected to the coil 101.

FIG. 4 is a cross-sectional elevated front view of the preferred embodiment of the TVEMF sleeve 10 depicted in FIG. 3. In FIG. 4 is shown the conductive portion 107 of the coil and the coil support 105 of the coil. The conductive portion 107 in this embodiment is an electrically conductive wire, preferably insulated, that is wrapped around the exterior portion of the coil support 105. The coil support 105 is preferably substantially rigid thereby maintaining and supporting the shape of the conductive portion 107. Also illustrated in FIG. 4 is a TVEMF source 103 that is operatively connected to the coil. The TVEMF source 103 in this preferred embodiment is removably affixed to the coil support 105. It can be affixed with any fastener known in the art including, but not limited to, hook and loop fasteners, for instance Velcro, and adhesives. Also depicted is a battery 104 contained within the TVEMF source 103. A battery operated TVEMF source of the TVEMF sleeve 10 provides a user with freedom of mobility so that the user is not required to remain close to an electrical outlet for the duration of the use.

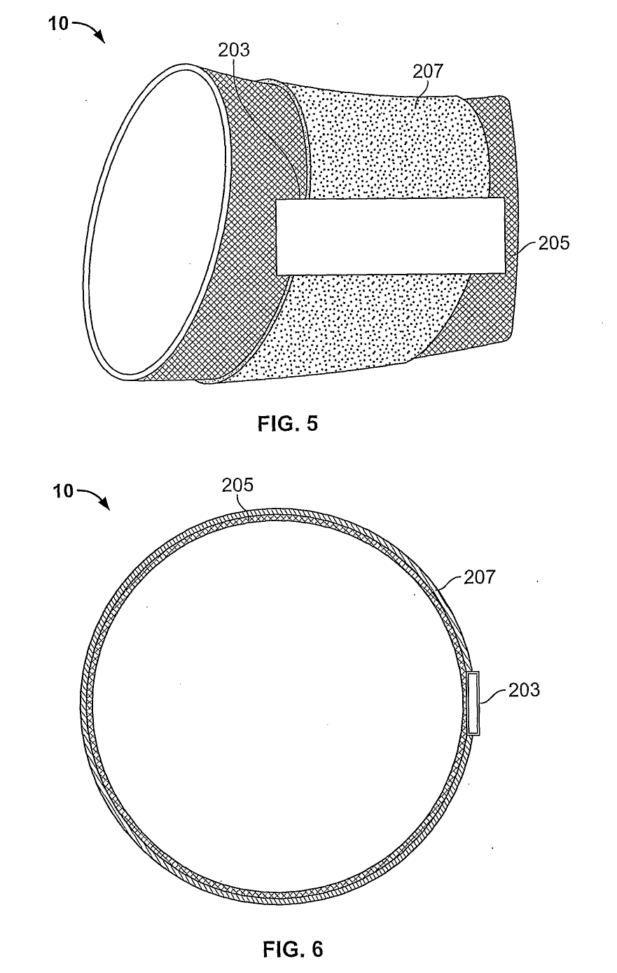

Illustrated in FIG. 5 is a side perspective of a TVEMF sleeve 10 having a TVEMF source 203 and a coil comprising a conductive portion 207 and a coil support 205. The coil support 205 of the coil has an interior portion and an exterior portion. The interior portion defines a space in which a mammalian body part can be removably received. The exterior portion of the coil support 205 is coated with the conductive portion 207, preferably a conductive metal, more preferably a ferromagnetic metal, and most preferably silver. The conductive portion 207 can preferably be sprayed onto the exterior portion of the coil support 205 in a substantially thin coat. The conductive portion 207 may also preferably be embedded within the coil support 205. The conductive portion 207 may also preferably be a substantially thin silver overlay. By the phrase “substantially thin,” it is intended that the conductive portion can facilitate a substantially flexible coil. Therefore, preferably a substantially flexible coil comprises a substantially thin conductive portion and a substantially flexible coil support, preferably non-conductive. It is further intended that a substantially thin conductive portion is not so thin that it cannot conduct a TVEMF. The coil support 205 preferably comprises a material that is substantially flexible, including, but not limited to, lycra, Dacron, and nylon. The TVEMF sleeve 10 in the preferred embodiment in FIG. 5 can be introduced to a mammalian body part and because of the substantially flexible coil, preferably comprising a substantially thin conductive portion 207 and a substantially flexible coil support 205, the TVEMF sleeve 10 can be comfortably used for extended periods of time. Furthermore, the TVEMF source 203 affixed, preferably removably, to the coil provides the user with freedom of movement. Moreover, the substantially flexible coil support 205, is thought to expand when introduced to a mammalian body part because of the preferrably substantially flexible coil support, and at the same time, the substantially thin conductive portion of the coil remains capable of conducting a TVEMF.

FIG. 6 is a cross-sectional elevated front perspective of the preferred embodiment of the TVEMF sleeve 10 illustrated in FIG. 5 comprising a coil and a TVEMF source 203 operatively connected to the coil. The coil further comprises a coil support 205 and a conductive portion 207. The preferred embodiment of the TVEMF sleeve 10 illustrated in FIGS. 5 and 6 can preferably further comprise a cover over the conductive portion, preferably a non-conductive cover.

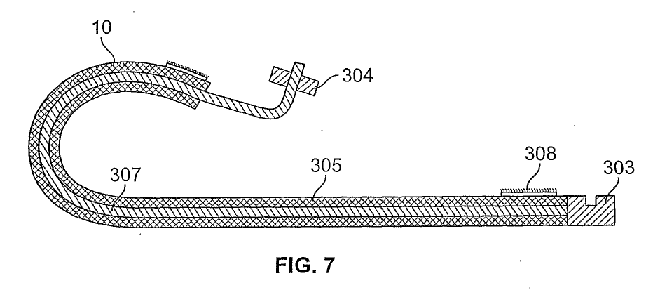

FIG. 7 illustrates yet another preferred embodiment of the TVEMF sleeve 10, a cross-sectional elevated side view, comprising a TVEMF source 303 operatively connected to a first end of the coil, and having a coil support 305 with an exterior, interior, and middle portion, and a conductive portion 307 contained within the middle portion of the coil support 305. The conductive portion 307 may preferably be a spray, more preferably an electrically conductive wire. Also depicted is a fastener 308 integral with the coil. The fastener 308 can be any fastener known in the art including, but not limited to, a hook and loop fastener and an adhesive. The second end of the coil has a coupling 304 for removably connecting the second end of the conductive portion 307 to the TVEMF source 303. In use, a mammalian body part having defective tissue is introduced to the TVEMF sleeve 10. The first and second end of the coil are wrapped around the mammalian body part and fastened together by the fastener 308. Thus, the interior portion of the coil defines a space that removably receives a mammalian body part.

In operation, a TVEMF sleeve is introduced to a mammalian body part with defective tissue. The phrase “mammalian body part,” and similar terms and phrases, is intended to preferably include, but is not limited to, a mammalian torso, head and limbs, preferably arms, legs, and/or a neck. Mammalian body parts may also preferably be the digits of the limbs, for instance, fingers and/or toes. The TVEMF source of the TVEMF sleeve is turned on and a TVEMF is delivered through the coil into defective tissue of a mammalian body part encompassed by the TVEMF sleeve. By “encompassed,” and similar terms, it is meant that the coil of the TVEMF sleeve surrounds the mammalian body part, and therefore, the defective tissue therein. The TVEMF sleeve is introduced to a mammalian body part and encompasses the same to deliver a TVEMF thereto. The term “delivering,” and similar terms is intended to mean supplying, providing, and/or exposing. For instance, the TVEMF source delivers a TVEMF through the coil of the TVEMF sleeve to the defective tissue in the mammalian body part. In use, the TVEMF is delivered to defective tissue in the mammalian body part for enhancing repair of the same. In the present invention, the term “defective tissue,” or any other term similar term is intended to include, but is not limited to muscle, skin, and bone.

Because the present invention provides a method for supplying a TVEMF to defective tissue of a mammalian body part, a TVEMF sleeve is so sized and configured to removably receive the mammalian body part having defective tissue so that a TVEMF can be delivered to the defective tissue. The TVEMF source of the TVEMF sleeve may generate a TVEMF preferably of from about 0.05 gauss to about 6 gauss, more preferably of from about 0.05 gauss to about 0.5 gauss, and most preferably about 0.5 gauss. The TVEMF is preferably in a pulsed square wave form (following a Fourier curve), more preferably in a differentiated square wave, and most preferably in a delta wave. Preferably, the pulsed square wave has a frequency of about 2 to about 25 cycles/second, more preferably about 5 to about 20 cycles/second, and for example about 10 cycles/second, and the conductive portion preferably has an RMS value of from about 1 to about 1000 mA, more preferably of from about 1 to about 10 mA, for example 6 mA. However, these parameters are not meant to be limiting to the TVEMF of the present invention, and as such, may vary based on other aspects of this invention. TVEMF may be measured by standard equipment, for instance an EN331 Cell Sensor Gauss Meter.

To enhance the effectiveness of the TVEMF sleeve and repair of the defective tissue of the mammalian body part, before the TVEMF sleeve is introduced to the mammalian body part, preferably 24 hours before, preferably a calcium supplement is administered to the mammal. Not to be bound by theory, a calcium supplement is thought to increase the amount of calcium ions in the defective tissue of the mammalian body part. The calcium supplement is preferably administered to the mammal to sustain a heightened level of calcium ions during the delivery of the TVEMF to the defective tissue of the mammalian body part, and administration is preferably continued until termination of administration is desired. Commonly available over the counter calcium supplements are preferred.

Also preferably, sodium zeolite A is administered to the mammal prior to the delivery of a TVEMF to the defective tissue of a mammalian body part, and preferably during the delivery of a TVEMF. Not to be bound by the theory, but sodium zeolite A is thought to be effective in ion exchange, thus enhancing the effective delivery of the TVEMF to the defective tissue in the mammalian body part. Preferably the sodium zeolite A is administered to the mammal in a range of from about 10 mg/kg body weight to about 20 g/kg body weight, more preferably from about 10 mg/kg body weight to about 10 g/kg body weight. The amount of sodium zeolite A administered to the mammal will preferably depend on the severity of the injury and amount of tissue needing repair. If administered to the mammal through the mammal's feed, the amount of sodium zeolite A is not to exceed about 5% total food weight. The sodium zeolite A can preferably be administered each day, more preferably two times a day. The sodium zeolite A can preferably be administered to the mammal in a form selected from tablets, lozenges, capsules, powders, dragees, aqueous or oily suspensions, syrups, elixirs, and aqueous solutions. The sodium zeolite A can preferably be administered orally or in any other suitable way. Sodium zeolite A administered in the form of tablets or capsules is preferably in an amount preferably 20 mg/tablet or capsule, more preferably 100 mg/tablet or capsule, most preferably 1000 mg/tablet or capsule, and even more preferably 5000 mg/tablet or capsule. Sodium zeolite A is preferably administered to the mammal prior to and during the delivery of the TVEMF to the defective tissue of the mammalian body part and continued until the defective tissue of the mammalian body part is repaired or preferably until termination of the administration is desired.

If two samples of mammals having simple leg fractures are selected, and the first sample is given standard treatment for the leg fracture, and the second sample is given both the standard treatment for the leg fracture as well as a TVEMF delivered to the site of the fracture through a TVEMF sleeve, it is expected that in those samples where a TVEMF is delivered to the leg fracture, substantially reduced healing times, by more than a quarter the time, will result.

As various changes could be made in TVEMF sleeves, as are contemplated in the present invention, without departing from the scope of the invention, it is intended that all matter contained herein be interpreted as illustrative and not limiting.

1. A time varying electromagnetic force sleeve comprising:
a. a coil having a conductive portion and a coil support having an outside portion wherein the conductive portion is wrapped around the outside portion of the coil support and is a solenoid, and wherein the coil has an interior portion that defines a space that removably receives a mammalian body part; and
b. a time varying electromagnetic force source operatively connected to the coil.

2. The time varying electromagnetic force sleeve as in claim 1, wherein the coil has a substantially elliptical cross section.

3. The time varying electromagnetic force sleeve as in claim 1, wherein the coil has a substantially oval cross section.

4. The time varying electromagnetic force sleeve as in claim 1, wherein the coil has a substantially circular cross section.

5. The time varying electromagnetic force sleeve as in claim 1, wherein the coil is a solenoid.

6. The time varying electromagnetic force sleeve as in claim 1, wherein the coil is substantially rigid.

7. The time varying electromagnetic force sleeve as in claim 1, wherein the coil is substantially flexible.

8. The time varying electromagnetic force sleeve as in claim 1, wherein the coil support is substantially rigid.

9. The time varying electromagnetic force sleeve as in claim 1, wherein the conductive portion comprises a ferromagnetic material.

10. The time varying electromagnetic force sleeve as in claim 1, wherein the conductive portion is silver.

11. The time varying electromagnetic force sleeve as in claim 1I wherein the conductive portion is electrically conductive wire.

12. The time varying electromagnetic force sleeve as in claim 1, wherein the conductive portion is insulated.

13. The time varying electromagnetic force sleeve as in claim 12, wherein the insulation is substantially rigid.

14. A method of enhancing repair of defective tissue of a mammal comprising the steps of:
c. providing a time varying electromagnetic force sleeve comprising an electromagnetic force source operatively connected to a coil having a conductive portion and a coil support having an exterior portion and an interior portion, wherein the conductive portion is a solenoid that is wrapped around the coil support, wherein the conductive portion is an electrically conductive wire, and wherein the interior portion of the coil support defines a space that removably receives a mammalian body part having defective tissue;
d. introducing the time varying electromagnetic force sleeve to the mammalian body part having defective tissue; and
e. delivering a time varying electromagnetic force to the mammalian body part having defective tissue for enhancing repair of the defective tissue.

15. A method of enhancing tissue repair as in claim 14, wherein the time varying electromagnetic force is in the form of a square wave (following a Fourier curve).

16. A method of enhancing tissue repair as in claim 14, wherein the time varying electromagnetic force is in the form of a differentiated square wave.

17. A method of enhancing tissue repair as in claim 14, wherein the time varying electromagnetic force is in the form of a delta wave.

18. A method of enhancing tissue repair as in claim 15, wherein the square wave is of from about 0.05 gauss to about 6 gauss.

19. A method of enhancing tissue repair as in claim 15, wherein the square wave is of from about 0.05 gauss to about 0.5 gauss.

20. A method of enhancing tissue repair as in claim 15, wherein the square wave is about 0.5 gauss.

21. A method of enhancing tissue repair as in claim 14, further comprising the step of administering a calcium supplement to the mammal.

22. A method of enhancing tissue repair as in claim 21, wherein the calcium supplement is administered prior to step c.

23. A method of enhancing tissue repair as in claim 21, wherein the calcium supplement is administered concurrently with step c.

24. A method of enhancing tissue repair as in claim 14, further comprising the step of administering sodium zeolite A to the mammal.

25. A method of enhancing tissue repair as in claim 24, wherein sodium zeolite A is administered prior to step c.

26. A method of enhancing tissue repair as in claim 24, wherein the sodium zeolite A is administered concurrently with step c.

27. A method of enhancing tissue repair as in claim 24, wherein the sodium zeolite A is administered in a range of from about 10 mg/kg body weight/day to about 20 g/kg body weight/day.

28. A method of enhancing tissue repair as in claim 14, wherein the time varying electromagnetic force is delivered to the defective tissue until the defective tissue is repaired.

29. A method of enhancing tissue repair as in claim 21 wherein the calcium supplement is administered until the defective tissue is repaired.

30. A method of enhancing tissue repair as in claim 24, wherein the sodium zeolite A is administered until the defective tissue is repaired.

31. A method of enhancing tissue repair as in claim 24, wherein the sodium zeolite A is less than about 5% total feed weight.

32. A method of enhancing tissue repair as in claim 14, wherein the time varying electromagnetic force sleeve is substantially flexible.

33. A method of enhancing tissue repair as in claim 14, wherein the time varying electromagnetic force sleeve is substantially rigid.

34. A time varying electromagnetic force sleeve comprising:
f. a coil having a conductive portion and a coil support wherein the conductive portion is embedded in the coil support and the coil has an interior portion that defines a space that removably receives a mammalian body part; and
g. a time varying electromagnetic force source operatively connected to the coil.

35. A method of enhancing repair of defective tissue of a mammal comprising the steps of:
h. providing a time varying electromagnetic force sleeve of claim 34 wherein the interior portion of the coil support defines a space that removably receives a mammalian body part having defective tissue;
i. introducing the time varying electromagnetic force sleeve to the mammalian body part having defective tissue; and
j. delivering a time varying electromagnetic force to the mammalian body part having defective tissue for enhancing repair of the defective tissue.

36. A time varying electromagnetic force sleeve comprising:
k. a coil having a conductive portion and a coil support wherein the conductive portion is a silver overlay on the coil support and the coil has an interior portion that defines a space that removably receives a mammalian body part; and
l. a time varying electromagnetic force source operatively connected to the coil.

37. A method of enhancing repair of defective tissue of a mammal comprising the steps of:
m. providing a time varying electromagnetic force sleeve of claim 35 wherein the interior portion of the coil support defines a space that removably receives a mammalian body part having defective tissue;
n. introducing the time varying electromagnetic force sleeve to the mammalian body part having defective tissue; and
o. delivering a time varying electromagnetic force to the mammalian body part having defective tissue for enhancing repair of the defective tissue.

38. A method of enhancing repair of defective tissue of a mammal comprising the steps of:
p. providing a time varying electromagnetic force sleeve having a coil and a time varying electromagnetic force source operatively connected to the coil, wherein the coil comprises a coil support, a conductive portion, and an interior portion wherein the interior portion defines a space that removably receives a mammalian body part having defective tissue;
q. introducing the time varying electromagnetic force sleeve to the mammalian body part having defective tissue; and
r. delivering a time varying electromagnetic force to the mammalian body part having defective tissue for enhancing repair of the defective tissue.

39. A method of enhancing tissue repair as in claim 38, further comprising the step of administering a calcium supplement to the mammal.

40. A method of enhancing tissue repair as in claim 39, wherein the calcium supplement is administered prior to step c.

41. A method of enhancing tissue repair as in claim 39, wherein the calcium supplement is administered concurrently with step c.

42. A method of enhancing tissue repair as in claim 38, further comprising the step of administering sodium zeolite A to the mammal.

43. A method of enhancing tissue repair as in claim 42, wherein sodium zeolite A is administered prior to step c.

44. A method of enhancing tissue repair as in claim 42, wherein the sodium zeolite A is administered concurrently with step c.

45. A method of enhancing tissue repair as in claim 42, wherein the sodium zeolite A is administered in a range of from about 10 mg/kg body weight/day to about 20 g/kg body weight/day.

46. A method of enhancing tissue repair as in claim 38, wherein the time varying electromagnetic force is delivered to the defective tissue until the defective tissue is repaired.

47. A method of enhancing tissue repair as in claim 39 wherein the calcium supplement is administered until the defective tissue is repaired.

48. A method of enhancing tissue repair as in claim 42, wherein the sodium zeolite A is administered until the defective tissue is repaired.

49. A method of enhancing tissue repair as in claim 42, wherein the sodium zeolite A is less than about 5% total feed weight.
